(19) United States
(12) Patent Application Publication
(10) Pub. No.: US 2011/0257721 A1
(43) Pub. Date: Oct. 20, 2011

(54) Prosthetic Heart Valves and Delivery Methods

(75) Inventor: Charles Tabor, St. Louis Park, MN (US)

(73) Assignee: Medtronic, Inc., Minneapolis, MN (US)

(21) Appl. No.: 13/086,845

(22) Filed: Apr. 14, 2011

Related U.S. Application Data
(60) Provisional application No. 61/324,379, filed on Apr. 15, 2010.

(51) Int. Cl. A61F 2/84; A61F 2/82

(52) U.S. Cl. 623/1.11; 623/1.24

(57) ABSTRACT

A method of remodeling a stented device and an adjacent a valve region of a patient, including the steps of implanting a stented device into a native valve region of a patient, providing a first remodeling ring on a portion of a delivery system, advancing the remodeling ring into an interior area of the implanted stented device with the delivery system, radially expanding the remodeling ring until it modifies at least one of an aspect of a shape of the interior area of the implanted stented device and an aspect of a shape of the valve region in which it is positioned, and removing the delivery system from the patient.

### PRIORITY CLAIM

This application claims the benefit of U.S. Provisional Patent Application Ser. No. 61/324,379 filed Apr. 15, 2010, and titled “PROSTHETIC HEART VALVES AND DELIVERY METHODS”, the entire disclosure of which is incorporated herein by reference.

### TECHNICAL FIELD

The present invention relates to prosthetic heart valves. More particularly, it relates to devices, methods, and delivery systems for percutaneously implanting prosthetic heart valves.

### BACKGROUND

Diseased or otherwise deficient heart valves can be repaired or replaced using a variety of different types of heart valve surgeries. Typical heart valve surgeries involve an open-heart surgical procedure that is conducted under general anesthesia, during which the heart is stopped while blood flow is controlled by a heart-lung bypass machine. This type of valve surgery is highly invasive and exposes the patient to a number of potentially serious risks, such as infection, stroke, renal failure, and adverse effects associated with use of the heart-lung machine, for example.

Recently, there has been increasing interest in minimally invasive and percutaneous replacement of cardiac valves. Such surgical techniques involve making a small opening in the skin of the patient into which a valve assembly is inserted in the body and delivered to the heart via a delivery device similar to a catheter. This technique is often preferable to more invasive forms of surgery, such as the open-heart surgical procedure described above. In the context of pulmonary valve replacement, U.S. Patent Application Publication Nos. 2003/0199971 A1 and 2003/0199963 A1, both filed by Tower, et al., describe a valved segment of bovine jugular vein, mounted within an expandable stent, for use as a replacement pulmonary valve. The replacement valve is mounted on a balloon catheter and delivered percutaneously via the vascular system to the location of the failed pulmonary valve and expanded by the balloon to compress the valve leaflets against the right ventricular outflow tract, anchoring and sealing the replacement valve. As described in the articles: “Percutaneous Insertion of the Pulmonary Valve”, Bonhoeffer, et al., Journal of the American College of Cardiology 2002; 39: 1664-1669 and “Transcatheter Implantation of a Bovine Valve in Pulmonary Position”, Bonhoeffer, et al., Circulation 2000; 102: 813-816, the replacement pulmonary valve may be implanted to replace native pulmonary valves or prosthetic pulmonary valves located in valved conduits.

Various types and configurations of prosthetic heart valves are used in percutaneous valve procedures to replace diseased natural human heart valves. The actual shape and configuration of any particular prosthetic heart valve is dependent to some extent upon the valve being replaced (i.e., mitral valve, tricuspid valve, aortic valve, or pulmonary valve). In general, the prosthetic heart valve designs attempt to replicate the function of the valve being replaced and thus will include valve leaflet-like structures used with either bioprostheses or mechanical heart valve prostheses. In other words, the replacement valves may include a valved vein segment that is mounted in some manner within an expandable stent to make a stented valve. In order to prepare such a valve for percutaneous implantation, the stented valve can be initially provided in an expanded or uncrimped condition, then crimped or compressed around the balloon portion of a catheter until it is as close to the diameter of the catheter as possible.

Other percutaneously delivered prosthetic heart valves have been suggested having a generally similar configuration, such as by Bonhoeffer, P. et al., “Transcatheter Implantation of a Bovine Valve in Pulmonary Position.” Circulation, 2000; 102:813-816, and by Cribier, A. et al. “Percutaneous Transcatheter Implantation of an Aortic Valve Prosthesis for Calcific Aortic Stenosis.” Circulation, 2002; 106:3006-3008, the disclosures of which are incorporated herein by reference. These techniques rely at least partially upon a frictional type of engagement between the expanded support structure and the native tissue to maintain a position of the delivered prosthesis, although the stents can also become at least partially embedded in the surrounding tissue in response to the radial force provided by the stent and balloons that are sometimes used to expand the stent. Thus, with these transcatheter techniques, conventional sewing of the prosthetic heart valve to the patient's native tissue is not necessary.

With regard to transcatheter valves that are delivered to the heart to replace the aortic valve, these valves often can include stents or frames and often rely on relatively high radial force to reshape the implantation area. However, these high radial force stents or frames can sometimes be difficult to accurately deploy due to the large amount stored energy that is released during deployment, which can cause the stent or frame to “jump” or move to an area that is different from the desired implantation area. In some cases, such high radial force stents or frames also can be relatively difficult to pull back into a sheath in order to relocate them within a patient once they are released or partially released from the delivery system. Thus, there is a desire to provide a replacement heart valve system that is an alternative to the use of high radial force stents or frames for reshaping an implantation area of a patient.

### SUMMARY

The replacement heart valves used in accordance with the invention each include a valve structure attached within an interior area of an expandable stent or frame, along with at least one remodeling ring and/or skirting ring that is at least partially positioned within the valved stent or frame. The stents that are used include a wide variety of structures and features that can be used alone or in combination with features of other stents of the invention. Many of the structures are compressible to a relatively small diameter for percutaneous delivery to the heart of the patient, and then are expandable either via removal of external compressive forces (e.g., self-expanding stents), or through application of an outward radial force (e.g., balloon expandable stents). The devices delivered by the delivery systems of the types described herein can be used to deliver stents, valved stents, or other interventional devices such as ASD (atrial septal defect) closure devices, VSD (ventricular septal defect) closure devices, or PFO (patent foramen ovale) occluders.

Methods for insertion of the replacement heart valves, remodeling rings, and skirting rings used in accordance with the invention include delivery systems that can maintain these compressible and expandable structures in their compressed state during their insertion and allow or cause the structures to expand once they are in their desired location. The methods of the invention may include implantation of the structures using either an antegrade or retrograde approach. Further, any of the structures may be rotatable in vivo to allow them to be positioned in a desired orientation.

In accordance with the invention, valved stents can be used with one or more auxiliary remodeling rings to create a circular orifice at the annular level, which can be useful to optimize pericardial valve functionality as well as prevent or minimize paravalvular leakage. These valved stents can provide for more accurate valve deployment, repositionability, and/or resheathing, while managing annular circularity and paravalvular leakage.

In one embodiment of the invention, a relatively low radial force stent or frame is first deployed into an annular region of a heart, such as an aortic annulus, for example. One or more remodeling rings and/or skirting rings can then be deployed within the inner region of the low radial force stent at the annular level (e.g., below the pericardial valve region) to modify an out-of-round annuli and/or to prevent or minimize paravalvular leakage. One or more remodeling rings can additionally or alternatively be deployed at the outflow region of the low radial force stent to provide reshaping of the valve and/or to prevent or minimize stent migration relative to the anatomy of the patient.

### BRIEF DESCRIPTION OF THE DRAWINGS

The present invention will be further explained with reference to the appended Figures, wherein like structure is referred to by like numerals throughout the several views, and wherein:

FIG. 1 is a schematic front view of a relatively low radial force stent with a valve attached within its inner area;

FIG. 2 is a schematic top view of the stent of FIG. 1 positioned within an out-of-round annular region of a patient;

FIG. 3 is a schematic front view of a remodeling ring positioned in an at least partially crimped configuration on a distal end of a delivery system;

FIG. 4 is a schematic front view of the delivery system and remodeling ring of FIG. 3 positioned relative to the interior area of the stent of FIG. 1;

FIG. 5 is a schematic front view of the remodeling ring of FIG. 3 deployed within the stent of FIG. 1;

FIG. 6 is a schematic top view of the remodeling ring of FIG. 3 deployed within the stent of FIG. 1;

FIG. 7 is a front schematic view of another exemplary embodiment of a low radial force frame with a valve attached within its inner area as it can be positioned relative to an exemplary out-of-round aortic annular region of a patient;

FIG. 8 is a bottom view of a portion of the frame and aortic annular region illustrated in FIG. 7;

FIG. 9 is a front schematic view of the frame of FIG. 7, also including a remodeling ring positioned in the aortic annular region;

FIG. 10 is a bottom view of a portion of the frame and aortic annular region illustrated in FIG. 9;

FIG. 11 is a front schematic view of a portion of the frame shown in FIG. 7 as it can be positioned relative to an exemplary out-of-round aortic annular region of a patient, and including a skirting ring positioned in the aortic annular region;

FIG. 12 is a front schematic view of the frame of FIG. 7 as it can be positioned relative to an aortic annular region of a patient, and including a remodeling ring positioned in the outflow region;

FIG. 13 is a front schematic view of the system of FIG. 12 and further including an additional remodeling ring positioned in the annular region of a patient's anatomy.

### DETAILED DESCRIPTION

As referred to herein, the prosthetic heart valves used in accordance with various devices and methods of heart valve delivery may include a wide variety of different configurations, such as a prosthetic heart valve having tissue leaflets or a synthetic heart valve having polymeric, metallic, or tissue-engineered leaflets, and can be specifically configured for replacing any heart valve. In addition, while much of the description herein refers to replacement of aortic valves, the prosthetic heart valves of the invention can also generally be used in other areas of the body, such as for replacement of native mitral, pulmonic, or tricuspid valves, for use as a venous valve, or to replace a failed bioprosthesis, such as in the area of an aortic valve or mitral valve, for example.

Although each of the stents or frames described herein typically includes leaflets attached within their internal areas, the leaflets are not shown in many of the illustrated embodiments for clarity purposes. In general, these structures include a number of strut or wire portions arranged relative to each other to provide a desired compressibility, strength, and leaflet attachment zone(s) to the heart valve. Other details on particular configurations of the stents of the invention are also described below; however, in general terms, stents of the invention are generally tubular support structures, and leaflets will be secured within each support structure to provide a valved stent. The leaflets can be formed from a variety of materials, such as autologous tissue, xenograph material, or synthetics, as are known in the art. The leaflets may be provided as a homogenous, biological valve structure, such as a porcine, bovine, or equine valve. Alternatively, the leaflets can be provided as independent structures (e.g., as can be formed with bovine or equine pericardial leaflets) and subsequently assembled to the support structure of the stent. In another alternative, the stent and leaflets can be fabricated at the same time, such as may be accomplished using high strength nano-manufactured NiTi films of the type produced at Advanced Bio Prosthetic Surfaces Ltd. (ABPS) of San Antonio, Tex., for example. The support structures are generally configured to accommodate three leaflets; however, the replacement prosthetic heart valves of the invention can incorporate more or less than three leaflets.

In more general terms, the combination of a support structure with one or more leaflets can assume a variety of other configurations that differ from those shown and described, including any known prosthetic heart valve design. In certain embodiments of the invention, the support structure with leaflets utilize certain features of known expandable prosthetic heart valve configurations, whether balloon expandable, self-expanding, or unfurling (as described, for example, in U.S. Pat. Nos. 3,671,979; 4,056,854; 4,994,077; 5,332,402; 5,370,685; 5,397,351; 5,554,185; 5,855,601; and 6,168,614; U.S. Patent Application Publication No. 2004/0034411; Bonhoeffer P., et al., “Percutaneous Insertion of the Pulmonary Valve”, Pediatric Cardiology, 2002; 39:1664-1669; Anderson H R, et al., “Transluminal Implantation of Artificial Heart Valves”, EUR Heart J., 1992; 13:704-708; Anderson, J. R., et al., “Transluminal Catheter Implantation of New Expandable Artificial Cardiac Valve”, EUR Heart J., 1990, 11: (Suppl) 224a; Hilbert S. L., “Evaluation of Explanted Polyurethane Trileaflet Cardiac Valve Prosthesis”, J Thorac Cardiovascular Surgery, 1989; 94:419-29; Block P C, “Clinical and Hemodyamic Follow-Up After Percutaneous Aortic Valvuloplasty in the Elderly”, The American Journal of Cardiology, Vol. 62, Oct. 1, 1998; Boudjemline, Y., “Steps Toward Percutaneous Aortic Valve Replacement”, Circulation, 2002; 105:775-558; Bonhoeffer, P., “Transcatheter Implantation of a Bovine Valve in Pulmonary Position, a Lamb Study”, Circulation, 2000:102:813-816; Boudjemline, Y., “Percutaneous Implantation of a Valve in the Descending Aorta In Lambs”, EUR Heart J, 2002; 23:1045-1049; Kulkinski, D., “Future Horizons in Surgical Aortic Valve Replacement: Lessons Learned During the Early Stages of Developing a Transluminal Implantation Technique”, ASAIO J, 2004; 50:364-68; the teachings of which are all incorporated herein by reference).

Orientation and positioning of the compressible and expandable structures of the invention may be accomplished either by self-orientation of the structures (such as by interference between features of the stent and a previously implanted stent or valve structure) or by manual orientation of the structure to align its features with anatomical or previous bioprosthetic features, such as can be accomplished using fluoroscopic visualization techniques, for example. In some embodiments, when aligning the structures of the invention with native anatomical structures, they should be aligned so as to not block the coronary arteries, and native mitral or tricuspid valves should be aligned relative to the anterior leaflet and/or the trigones/commissures.

The various support structures described herein can be a series of wires or wire segments arranged so that they are capable of transitioning at least once, and preferably multiple times, from a collapsed state to an expanded state. In some embodiments, a number of individual wires comprising the support structure can be formed of a metal or other material. These wires are arranged in such a way that the support structure can be folded or compressed to a contracted state in which its internal diameter is at least somewhat reduced from its internal diameter in an expanded state. In its collapsed state, such a support structure with an attached valve can be mounted over a delivery device, such as a balloon catheter, for example. The support structure is configured so that it can be changed to its expanded state when desired, such as by the expansion of a balloon catheter that presses outward in a radial direction against the support structure. The delivery systems used for such a support structure should be provided with degrees of rotational and axial orientation capabilities in order to properly position the new stent at its desired location.

The wires of the support structure of the stents in other embodiments can instead be formed from a shape memory material such as a nickel titanium alloy (e.g., Nitinol) or a very high-tensile material that will expand from its compressed state to its original state after removal of external forces. With this material, the support structure is self-expandable from a contracted state to an expanded state, such as by the application of heat, energy, and the like, or by the removal of external forces (e.g., compressive forces that are provided by a sheath or other holding structure). This support structure can preferably be repeatedly compressed and expanded without damaging the structure of the stent. In addition, the support structure of such an embodiment may be laser cut from a single piece of material or may be assembled from a number of different components. For these types of structures, one example of a delivery system that can be used includes a catheter with a retractable sheath that covers the support structure until it is to be deployed, at which point the sheath can be retracted to allow the stent to expand. Alternatively, the support structures of the invention can be implanted using conventional surgical techniques and/or minimally invasive surgical procedures. In such cases, the support structures of the invention can advantageously require relatively few or no sutures to secure the stent to an anatomical location within the patient.

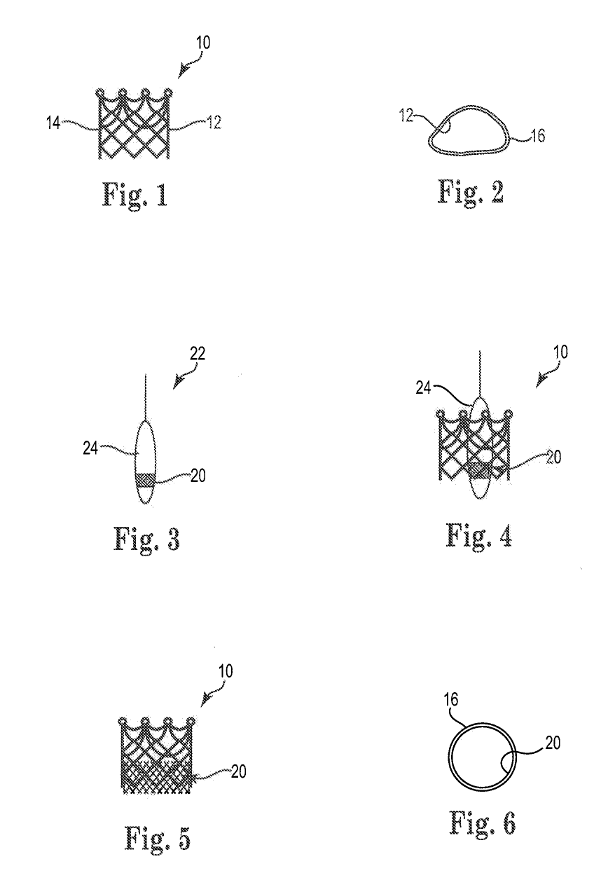

Referring now to the Figures, wherein the components are labeled with like numerals throughout the several Figures and initially to FIGS. 1-6, an embodiment of a replacement heart valve system of the invention is illustrated. Such a system can be used to position a replacement heart valve into a native valve space in a patient, where the interior shape of the native valve opening is different from the outer shape of the valve when it is initially positioned within the patient. In accordance with an aspect of the invention, in order to properly implant the replacement heart valve within the patient, the interior shape of the valve opening will be modified at least slightly so that it matches or closely matches the outer shape of the replacement valve.

FIGS. 1 and 2 illustrate an exemplary embodiment of a valved stent 10, which includes a relatively low radial force stent or support structure 12 having a valve 14 attached within its inner area. As shown in this embodiment, stent 12 includes a series of zig-zag ring structures that are coupled longitudinally to one another to form a generally cylindrical-shaped structure, although it is understood that the structures can be arranged in an at least slightly oval or elliptical shape. Each ring structure takes the form of a series of adjacent generally straight sections which each meet one another at one end at a curved or angled junction to form a generally “V” or “U” shaped structure. Stent 12 can be fabricated using wire stock, for example, or may instead be produced by machining the stent from a metal tube, as is commonly employed in the manufacturing of stents. The number of wires, the positioning of such wires, and various other features of the stent chosen can vary considerably from that shown in FIG. 1. Thus, the specifics of the stent can vary widely, such that many other known generally cylindrical stent configurations may be used within the scope of the invention. In accordance with the invention, the stent 12 is designed or chosen to have a relatively low outward radial force when deployed, as will be discussed in further detail below.

FIG. 2 illustrates the stent or support structure 12, without its leaflets or valve structure 14, as it can be positioned within a native, irregularly shaped annular region 16 of a patient. Region 16 is shown schematically in this figure to be an irregular oval or elliptical shape; however, it is understood that the region in which the stent is implanted can have a different shape. Stent 12 is shown in its expanded or partially expanded condition, as such a stent may have been delivered by a delivery system, such as a transcatheter valve delivery system of the type described herein, for example. The stent structure can be in this condition as it has expanded due to the removal of an external force (e.g., a self-expanding stent having an outer sheath removed) or as it has been expanded with the application of an outward radial force (e.g., a balloon that has been inflated within its internal area), for example. As illustrated in this figure, the outward radial force provided by the stent 12 is designed to not significantly change the shape of the annular area 16 after it has been delivered to the annular area, since it provides a relatively low radial force. That is, at this point in the process, the stent 12 does not provide sufficient radial force to correct or reshape the out-of-round anatomy of the patient in the implantation area.

In order to modify or remodel the shape of the implantation area of the patient in accordance with the invention, a remodeling ring 20 is mounted onto a distal end of a delivery system 22, as is illustrated in FIG. 3. The remodeling ring 20 is capable of providing enough outward radial force to reshape the annular area of the patient in the location in which it is deployed. As shown in this embodiment, the remodeling ring 20 has a height that is considerably smaller than the overall height of the stent 12 in which it will be positioned; however, this difference in the heights is only intended to be exemplary. The ring 20 can instead have a height that is closer to that of the stent in which it will be positioned, and can even have a greater height than the height of the stent in which it will be positioned, if desired. The remodeling ring 20 can have a generally mesh-like structure, as shown, or can instead have another structure that is expandable to take on a desired shape and size when deployed within the patient. For another example, the remodeling ring can have areas that are solid or semi-solid to provide larger sections of the ring material that will be in contact with the inner area of the stent in which it is positioned.

In this exemplary embodiment, the distal region of the delivery system 22 that is illustrated includes a balloon 24 that is expandable to provide outward radial force to a balloon expandable support structure, such as remodeling ring 20. However, it is contemplated that the remodeling ring may instead be a self-expanding remodeling ring, wherein the delivery system can then include a sheath or other structure to maintain the remodeling ring in a compressed condition until it is desired to allow it to expand outwardly. At this point, the sheath or other holding structure can be removed or retracted from the remodeling ring to allow it to expand outwardly.

The delivery system 22 can further include a proximal end with one or more control mechanisms to guide the distal region on which the remodeling ring 20 is mounted to the desired expansion area, along with control mechanisms to inflate and deflate the balloon 24. The delivery system may also include a guide wire or other component to assist in locating the proper position for deployment of the ring 20.

The delivery system 22 is used to maneuver the remodeling ring 20 into the inner region of the stent 12, as is generally illustrated in FIG. 4. As is illustrated in this embodiment, the remodeling ring 20 can be located below the area (e.g., annular region 16) where the valve is attached within the stent 12 in order to not interfere with the performance of the valve. The remodeling ring 20 is then expanded outwardly by inflating the balloon 24, as is illustrated in FIG. 5, until it modifies or reshapes the anatomy in this area of the patient. The reshaping of the annular region of the patient can continue until it takes on a certain desired shape, such as the circular shape illustrated in FIG. 6. At this point, the balloon 24 can be deflated and the delivery system 22 can be removed from the patient.

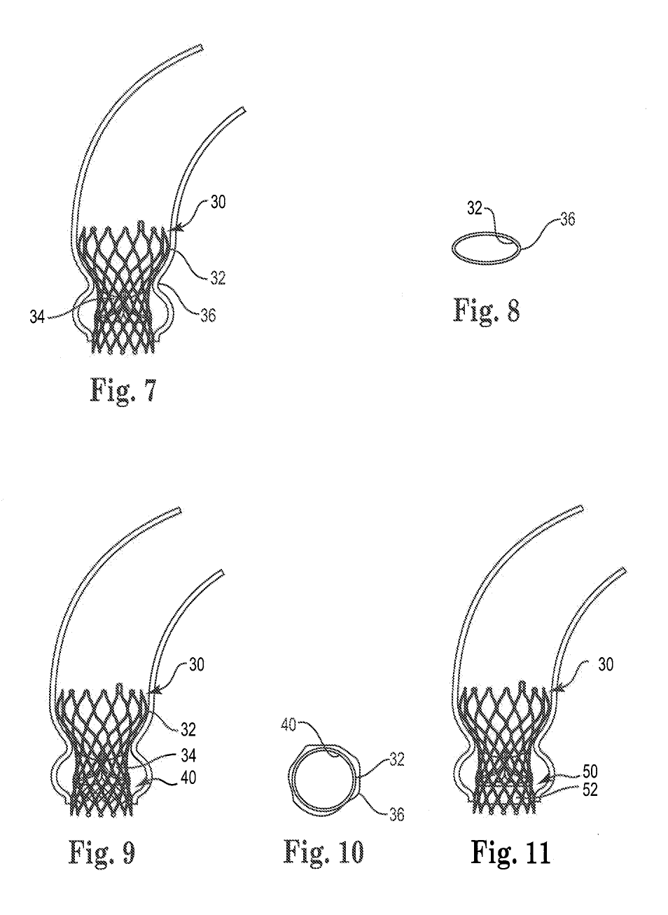

FIG. 7 is a schematic view of an exemplary embodiment of another version of a valved frame 30 as it is being positioned within an area of a patient's anatomy. Valved frame 30 includes a relatively low radial force frame 32 with a valve 34 attached within its inner area. As illustrated in the exemplary bottom view of FIG. 8, valved frame 30 is positioned within a native aortic annular region 36 of a patient, which is illustrated as being generally oval or elliptical in shape. Because such a shape of the native anatomy can allow for paravalvular leakage and/or prevent optimal performance of the implanted valve 34, which is generally circular in shape when expanded, a remodeling ring 40 of the invention can be used. In particular, remodeling ring 40 can be positioned within the implanted valve 34 in the annular region of the anatomy of the patient, as is illustrated in FIG. 9. As the ring 40 is expanded, it reshapes the annular region 36 of the patient to be circular or relatively circular, as is illustrated in FIG. 10. In this way, the valved frame 30 will more closely fit within the annular region 36, thereby minimizing the chances of paravalvular leakage.

FIG. 11 illustrates a front schematic view of the valved frame 30 of FIG. 7 with a skirting ring 50 positioned within its internal area within the annular region of the patient. Skirting ring 50 includes a frame structure (not visible) along with a material covering 52. As with the auxiliary support members described above (e.g., remodeling ring 40), the skirting ring 50 can also provide sufficient radial force to reshape the area in the patient where it is implanted. When a skirting ring or remodeling ring is provided by a separate component as described herein, it is not necessary to implant the valved frame 30 with the skirt via a single delivery device, thereby allowing for a smaller overall crimped size for a valved frame when it is originally implanted.

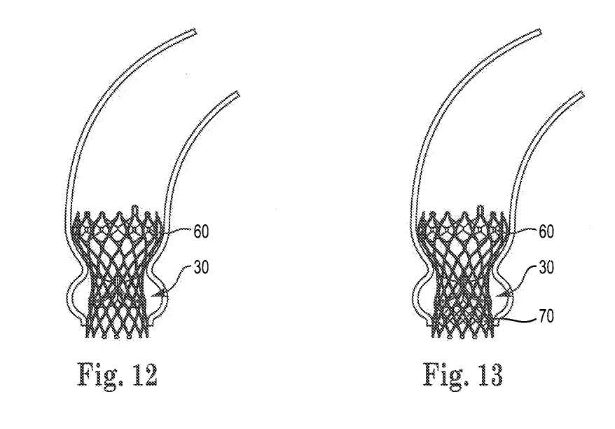

FIG. 12 is a front view of the valved frame 30 of FIG. 7 and including a remodeling ring 60 positioned in the outflow region of an aorta rather than the annular region that is described above. The remodeling ring 60 can also provide for remodeling or reshaping of the frame 30, as described above relative to other remodeling rings or skirting rings. However, the remodeling ring 60 provides additional anchoring for the valved frame 30, thereby preventing or minimizing undesirable migration of the valved frame within the patient's anatomy. FIG. 13 illustrates the embodiment of FIG. 12, with an additional remodeling ring 70 positioned generally adjacent the annular region for reshaping of the annular region, as described above. These locations for remodeling rings are only provide a limited number of exemplary locations that are contemplated by the current invention. That is, it is possible to provide only a single remodeling ring positioned at any area along the height of a particular stented frame in which it is desired to remodel or reshape the native anatomy, and it is further within the scope of the invention to provide one or more additional remodeling rings within that same stented frame when it is desired to remodel or reshape multiple areas of the native anatomy. In this way, a desired fit between the anatomy and the device can be achieved.

When more than one remodeling ring is used within one stented frame, the multiple rings can either be delivered sequentially or simultaneously. If the rings are delivered sequentially, they can be the same or different from each other, and can be delivered using the same or different delivery systems. Different delivery systems may be required if the amount of required expansion for the multiple remodeling rings is substantially different. If the rings are delivered simultaneously, it is further contemplated that they can be expanded simultaneously, such as with multiple balloons and inflation systems that are independently controllable or with a single balloon that is controllable for simultaneous inflation of the rings with a single inflation controller. It is also contemplated that the balloon and remodeling ring(s) can be positioned on their own delivery system or that the balloon can be positioned on the same delivery system that is delivering the stented valve to the patient.

Although the above description generally includes exemplary embodiments that include one remodeling ring and/or skirting ring positioned within a particular valved frame structure, it is understood that more than one remodeling ring and/or skirting ring may be used with a valve frame structure, where the multiple rings can be adjacent to each other in a radial direction, a longitudinal direction relative to the length of the frame structure, or in some other arrangement. Each of the multiple remodeling rings may have similar or different properties from each other. For one example, remodeling rings of increasing radial force capabilities can be implanted within each other in cases where the desired reshaping of the region of the patient is not achieved with deployment of a single remodeling ring.

The remodeling rings and the delivery systems and methods of the invention can advantageously be used in cooperation with many other types of delivery systems for a wide variety of stents positioned in various locations in a patient. In this way, if it is determined at the time of a stent implantation or at a later date that the implanted stent should be expanded further in order to remodel the area in which it is implanted, it is possible to then utilize the delivery systems and remodeling rings of the invention.

The present invention has now been described with reference to at least one embodiment thereof. The contents of any patents or patent application cited herein are incorporated by reference in their entireties. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

What is claimed is:

1. A method of remodeling a stented device and an adjacent valve region of a patient, comprising the steps of:
implanting a stented device into a native valve region of a patient;
providing a first remodeling ring on a portion of a delivery system;
advancing the remodeling ring into an interior area of the implanted stented device with the delivery system;
radially expanding the remodeling ring until it modifies at least one of an aspect of a shape of the interior area of the implanted stented device and an aspect of a shape of the valve region in which it is positioned; and
removing the delivery system from the patient.

2. The method of claim 1, wherein the stented device comprises:
a support frame having an interior area, and
a valve attached to the support frame within the interior area.

3. The method of claim 1, wherein the portion of the delivery system on which the first remodeling ring is positioned comprises a balloon, and wherein the step of radially expanding the remodeling ring comprises the step of radially expanding the balloon within the remodeling ring.

4. The method of claim 1, wherein the portion of the delivery system on which the first remodeling ring is positioned comprises an external sheath, and wherein the step of radially expanding the first remodeling ring comprises the step of axially sliding the sheath relative to the first remodeling ring to allow the first remodeling ring to radially expand.

5. The method of claim 2, wherein the step of advancing the first remodeling ring into the interior area of the implanted stented device further comprises positioning the remodeling ring adjacent to the valve within the interior area of the support frame.

6. The method of claim 1, wherein the native valve region comprises an annular valve region of a patient.

7. The method of claim 6, wherein the annular valve region comprises an annulus of a native aortic valve.

8. The method of claim 1, wherein the first remodeling ring comprises a mesh tubular structure.

9. The method of claim 1, wherein the first remodeling ring comprises a frame structure and a covering over at least a portion of the frame structure.

10. The method of claim 1, wherein the radial expansion of the remodeling ring modifies the size of the valve region.

11. The method of claim 1, wherein the step of radially expanding the remodeling ring comprises modifying both an aspect of a shape of the interior area of the implanted stented device and an aspect of a shape of the valve region in which it is positioned.

12. The method of claim 1, further comprising the steps of:
providing a second remodeling ring on a portion of a delivery system;
advancing the second remodeling ring into an interior area of the implanted stented device and spaced from the first remodeling ring with the delivery system; and
radially expanding the second remodeling ring until it modifies at least one of an aspect of a shape of the interior area of the implanted stented device and an aspect of a shape of the valve region in which it is positioned.

13. The method of claim 12, wherein the first and second remodeling rings are delivered into the interior area of the implanted stented device with the same delivery system.

14. The method of claim 12, wherein the first and second remodeling rings are radially expanded sequentially.

15. The method of claim 12, wherein the first and second remodeling rings are radially expanded simultaneously.

16. The method of claim 12, wherein the first remodeling ring has at least one different material property from the second remodeling ring.

17. A method of remodeling a stented device and an adjacent valve region of a patient, comprising the steps of:
implanting a low-radial force stented device into a native valve region of a patient;
providing a first remodeling ring on an expandable portion of a delivery system;
advancing the remodeling ring into an interior area of the implanted stented device with the delivery system;
expanding the expandable portion of the delivery system to cause the remodeling ring to radially expand until it modifies at least one aspect of a shape of the interior area of the implanted stented device and also modifies at least one aspect of a shape of the valve region in which it is positioned; and
removing the delivery system from the patient.

18. The method of claim 17, further comprising the steps of:
providing a second remodeling ring on an expandable portion of a delivery system;
advancing the second remodeling ring into an interior area of the implanted stented device and spaced from the first remodeling ring; and
expanding the expandable portion of the delivery system to cause the second remodeling ring to radially expand until it modifies at least one of an aspect of a shape of the interior area of the implanted stented device and an aspect of a shape of the valve region in which it is positioned.

19. The method of claim 18, wherein at least one of the first and second remodeling rings comprises a mesh tubular structure.

20. The method of claim 18, wherein at least one of the first and second remodeling rings comprises a frame structure and a covering over at least a portion of the frame structure.
